SEQUENCE LISTING (2 sequences)

SEQ ID NO: 1 - DNA, 23 bp, Artificial Sequence
  Description of Artificial Sequence PRIMER
gcaaaactctatggtcctgatgt

SEQ ID NO: 2 - DNA, 21 bp, Artificial Sequence
  Description of Artificial Sequence PRIMER
gcaaaggtgtcggatagcaag

(19) United States
(12) Patent Application Publication
(10) Pub. No.: US 2005/0192209 A1
(43) Pub. Date: Sep. 1, 2005

(54) Eosinophil Major Basic Protein as a natural heparanase-inhibiting protein, compositions, methods and uses thereof

(76) Inventors: Israel Vlodavsky, Mevasseret Zion (IL); Gerald J. Gleich, Salt Lake City, UT (US); Francesca Levi-Schaffer, Jerusalem (IL)

Correspondence Address: FLEIT KAIN GIBBONS GUTMAN BONGINI & BIANCO, COURVOISIER CENTRE II, SUITE 404, 601 BRICKELL KEY DRIVE, MIAMI, FL, 33131, US

(21) Appl. No.: 10/789,428

(22) Filed: Feb. 27, 2004

(51) Int. Cl. A61K 38/17; A61K 38/54

(52) U.S. Cl. 514/2; 514/12

(57) ABSTRACT

The use of a eosinophil secondary granules basic protein and any functional fragments thereof, and preferably, the use of Major Basic Protein (MBP) purified from eosinophils, as a natural heparanase inhibitor. Also, methods for the inhibition of the catalytic activity of heparanase using MBP, and to methods and compositions for the treatment of heparanase associated pathologic disorders. These methods and composition are particularly useful for the treatment of angiogenesis, tumor formation, tumor progression or tumor metastasis.

### BACKGROUND OF THE INVENTION

1. Field of the Invention

The invention relates to natural heparanase inhibitors, and uses thereof in the treatment of pathologic disorders and processes associated with heparanase glycosidase catalytic activity. More particularly, the invention relates to the use of a eosinophil secondary granules basic protein and any functional fragments thereof, and preferably, the use of Major Basic Protein (MBP), as a heparanase inhibitor. The invention further relates to the use of MBP in the preparation of compositions and methods for the treatment of heparanase associated pathologic disorders.

2. Prior Art

Throughout this application various publications are referenced to. It should be appreciated that the disclosure of these publications in their entireties are hereby incorporated into this application in order to more fully describe the state of the art as known to those skilled therein as of the date of the invention described and claimed herein.

Eosinophils participate in allergic inflammation after infiltration from the peripheral blood into the tissue. In the inflamed tissue, eosinophils have been historically thought to cause damage mostly through the release of the granule-associated cationic proteins Major Basic Protein (MBP), Eosinophil Cationic Protein (ECP), Eosinophil Peroxidase (EPO) and Eosinophil Derived Neurotoxin (EDN) [Gleich, G. J. J. Allergy Clin. Immunol. 105:651-3 (2000)]. MBP, localized in the core of the eosinophil secondary granules and comprising over 50% of the granule protein, appears to play a role in host defense as well as in tissue damage [Gleich (2000) ibid.]. Many of the biological properties of MBP have been attributed to the strong positive charge of the molecule and its high arginine content [O'Donnell, M. C. et al., J. Exp. Med. 157:1981-91 (1983)], even though its cationic nature does not fully explain its activity [Thomas, L. L. et al., Immunol. Lett. 78:175-81 (2001)]. More recently, however, a tissue-healing role has been ascribed to eosinophils and attributed to their ability to produce pro-fibrogenic cytokines, metalloproteinases and tissue inhibitors of metalloproteinases [Levi-Schaffer, F. et al., Proc. Natl. Acad. Sci. USA 96:9660-5 (1999)]. In addition to allergy, eosinophils are associated with a number of chronic inflammatory and malignant diseases [Gleich et al., (2000) ibid.; Samoszuk M. Histol Histopathol 12:807-12 (1997)]. This and the increasing significance ascribed to heparanase in inflammation, angiogenesis, wound healing and cancer progression [Dempsey, L. A. et al., Trends Biochem. Sci. 25:349-51 (2000); Vlodavsky, I. and Friedmann, J. Clin. Invest. 108:341-7 (2001)], prompted the present inventors to investigate the presence of heparanase in eosinophils.

Heparanase exerts its biological effects primarily through specific intra-chain cleavage of heparan sulfate (HS) [Pikas, D. S. et al., J. Biol. Chem. 273:18770-7 (1998)] and release of extracellular matrix (ECM)-resident heparin-binding growth and differentiation factors [Vlodavsky (2001) ibid.; Vlodavsky, I. et al., Trends Biochem. Sci. 16:268-71 (1991)].

HS degradation by mammalian endoglycosidic enzymes was first described in human placenta and rat liver hepatocytes. Since then, heparanase activity has been identified in a variety of normal and malignant cells and tissues [Vlodavsky, I. et al., Invasion & Metastasis 12:112-127 (1992); Parish, C. R. et al., Biochim. Biophys. Acta. 1471:M99-M108 (2001); Vlodavsky, I. and Friedmann, Y. J. Clin. Invest. 108:341-347 (2001); Nakajima, M. et al., J. Cell. Biochem. 36:157-163 (1988); Bernard, D. et al., J. Invest. Dermatol. 117:1266-73 (2001)]. Heparanase cleaves the glycosidic bond with a hydrolase mechanism and is thus distinct from bacterial heparinases and heparitinase, which are called eliminases, to indicate their ability to remove the polysaccharide from the core protein in a single step. HS glycosaminoglycan chains are cleaved by heparanase at only few sites, yielding HS fragments of appreciable size (10-20 sugar units) [Pikas, D. S. et al., J. Biol. Chem. 273:18770-18777 (1998)].

Heparanase is synthesized as a latent protein of 65 kDa that is processed at the N-terminus into an active 50 kDa form [Fairbanks, M. et al., J. Biol. Chem. 274:29587-90 (1999); Hulett, M. D. et al., Nat. Med. 5:803-9 (1999); Vlodavsky, I. et al., Nat. Med. 5:793-802 (1999 (a))].

The heparanase cDNA contains an open reading frame of 1629 bp encoding a 61.2 kDa polypeptide of 543 amino acids. The mature active 50 kDa enzyme, isolated from cells and tissues, has its N-terminus 157 amino acids downstream from the initiation codon, suggesting post-translational processing of the heparanase polypeptide at an unusual cleavage site (Gln¹⁵⁷-Lys¹⁵⁸) [Vlodavsky (1999 (a)) ibid.; Hulett, M. D. et al., Nat. Med. 5:803-809 (1999); Fairbanks, M. B. et al., J. Biol. Chem. 274:29587-29590 (1999)].

The fact that highly homologous cDNA sequences were derived from different species and types of normal and malignant cells is consistent with the notion that one dominant functional HS degrading endoglycosidase is expressed by mammalian cells [Parish, C. R. (2001) ibid.; Vlodavsky. and Friedmann (2001) ibid.]. Thus, unlike the large number of proteases that can solubilize polypeptides in the ECM, one major heparanase appears to be used by cells to degrade the HS side chains of HSPGs.

Because of the potential tissue damage that could result from inadvertent cleavage of HS, heparanase must be tightly regulated, although little is known about the control of its expression, activity, or subcellular localization. The enzyme is synthesized as a pro-enzyme and is localized mostly in perinuclear acidic endosomal and lysosomal granules of fibroblasts and tumor cells and in the tertiary granules of human neutrophils, where it is co-localized with MMP-9 [Nadav, L. et al., J. Cell Sci. 115(10):2179-87 (2002); Mollinedo, F. et al., Biochem. J. 327:917-923 (1997)]. Several observations suggest that heparanase can be membrane-bound. Heparanase immunoreactivity is observed on the surface of various normal and malignant cells [Vlodavsky and Friedmann (2001) ibid.; Friedmann, Y. et al., Am. J. Pathol. 157:1167-1175 (2000)]. The heparanase sequence contains a putative hydrophobic transmembrane domain [Hulett et al., (2000) ibid.] and its complete solubilization from rat liver, platelets and tumor cells, requires the presence of a detergent, indicating association with the cell membrane [Hulett (1999) ibid.].

Apart from tumor cells [Vlodavsky (2001) ibid.], heparanase activity is found primarily in platelets and activated cells of the immune system [Vlodavsky, I. et al., Invasion Metastasis 12:112-27 (1992); Matzner, Y. et al., J. Clin. Invest. 76:1306-13 (1985)].

Expression of heparanase was found to correlate with the metastatic potential of mouse lymphoma [Vlodavsky, I. et al., Cancer Res. 43:2704-2711 (1983)], fibrosarcoma and melanoma cells [Nakajima, M. et al., J. Cell. Biochem. 36:157 (1988)]. Similar correlation was observed in human breast, colon, bladder, prostate, and liver carcinomas [Vlodavsky (1999 (a)) ibid.]. Moreover, elevated levels of heparanase were detected in sera of metastatic tumor bearing animals [Nakajima (1988) ibid.] and of cancer patients, in urine of highly metastatic patients [Vlodavsky, I. et al., In: Tumor Angiogenesis. Eds. C. E. Lewis, R. Bicknell & N. Ferrara. Oxford University Press, Oxford UK, pp. 125 (1997)], and in tumor biopsies [Vlodavsky, I. et al., Isr. J. Med. 24:464 (1988)]. Treatment of experimental animals with heparanase substrates or inhibitors (e.g., non-anticoagulant species of low molecular weight heparin and polysulfated saccharides) considerably reduced the incidence of lung metastases induced by B16-F10 melanoma, Lewis lung carcinoma, and mammary adenocarcinoma cells [Vlodavsky, I. et al., Invasion Metastasis 14:290 (1994); Nakajima (1988) ibid.; Parish, C. R. et al., Int. J. Cancer 40:511 (1987); Lapierre, F. et al., Glycobiol. 6:355 (1996)], indicating that heparanase inhibitors may inhibit tumor cell invasion and metastasis.

Heparanase was also shown to be involved in primary tumor angiogenesis. Most primary solid tumors (1-2 mm diameter) obtain their oxygen and nutrient supply through a passive diffusion from pre-existing blood-vessels, however the increase in their mass beyond this size requires angiogenesis. Heparin-binding polypeptides such as vascular endothelial growth factor (VEGF) and basic fibroblast growth factor (bFGF) are highly mitogenic for vascular endothelial cells, and are among the most potent inducers of angiogenesis. bFGF has been extracted from the subendothelial ECM produced in vitro, and from basement membranes of cornea, suggesting that ECM may serve as a reservoir for bFGF.

Immunohistochemical staining revealed the localization of bFGF in basement membranes of diverse tissues and blood vessels. bFGF binds to HSPG in the ECM and can be released in an active form by HS-degrading enzymes. Heparanase expressed by platelets, mast cells, neutrophils, and lymphoma cells was found to be involved in the release of active bFGF from ECM and basement membranes, suggesting that heparanase activity may not only function in cell migration and invasion, but may also elicit an indirect neovascular response [Elkin (2001) ibid.].

Still further, it was shown that heparanase catalytic activity correlates with the ability of activated cells of the immune system to leave the circulation and elicit both inflammatory and autoimmune responses. Interaction of platelets, granulocytes, T and B lymphocytes, macrophages, and mast cells with the subendothelial ECM is associated with degradation of HS by heparanase [Vlodavsky, I. et al., Invasion Metastasis 12:112 (1992)]. The enzyme is released from intracellular compartments (e.g., lysosomes, specific granules) in response to various activation signals (e.g., thrombin, calcium ionophore, immune complexes, antigens, mitogens), suggesting its regulated involvement in inflammatory sites and in autoimmune diseases.

Indeed, treatment of experimental animals with heparanase substrates (e.g., low molecular weight heparin) markedly reduced the incidence of experimental autoimmune encephalomyelitis (EAE), adjuvant arthritis and graft rejection, indicating that heparanase inhibitors may inhibit autoimmune and inflammatory diseases [Lider, 0. J. Clin. Invest. 83:752 (1989)].

Heparanase inhibitors have been further proposed for treatment of human metastasis, for example, derivatives of siastatin B [Nishimura, Y. et al., J. Antibiot. 47:101 (1994); Kawase, Y. J. Antibiotics 49:61 (1995)], suramin, a polysulfonated naphthylurea [Nakajima, M. J. Biol. Chem. 266:9661 (1991)], sulfated oligosaccharides, e.g., sulfated maltotetraose and maltohexaose [Parish, C. R. et al., Cancer Res. 59:3433 (1999)], and sulfated polysaccharides [Parish (1987) ibid.; Lapierre (1996) ibid.].

U.S. Pat. No. 6,190,875 discloses methods of screening for agents inhibiting heparanase catalytic activity and hence potentially inhibiting tumor metastasis, autoimmune and inflammatory diseases which comprises interacting a native or recombinant heparanase enzyme with a heparin substrate in the presence or absence of a potential active agent and determining the inhibitory effect of said agent on the catalytic activity of said heparanase enzyme towards said heparin substrate.

WO 02060374 and WO92969867 disclose benz-1,3-azole and carbazole derivatives, respectively, and their use as heparanase inhibitors. However, none of the prior art heparanase inhibitors are natural proteins which endogenously exhibit their heparanase inhibitory activity.

There is thus an existing need for providing natural inhibitors, methods and compositions for inhibiting heparanase glycosidase catalytic activity.

### SUMMARY OF THE INVENTION

The present application describes for the first time a natural heparanase inhibitor protein. As demonstrated by the present invention, eosinophils that produce heparanase fail to express heparanase enzymatic activity in contrast to other cells. Detailed examination of this phenomenon indicated that basic proteins associated with eosinophil secondary granules, and particularly MBP, exhibit potent inhibitor of heparanase activity.

Therefore, a further object of the invention is to provide compositions comprising eosinophil secondary granules basic proteins, preferably MBP, and methods for the inhibition of heparanase and for the treatment of any heparanase associated disorder. These compositions and methods are particularly applicable for the treatment and the inhibition of processes and pathologies shown as involving heparanase catalytic activity.

These and other objects of the invention will become apparent as the description proceeds.

The present invention relates to a composition for the inhibition of heparanase glycosidase catalytic activity. The invention further provides a pharmaceutical composition for the treatment or the inhibition of a process or a pathologic disorder associated with heparanase catalytic activity. According to a specific embodiment, the compositions of the invention comprise as an active ingredient any one of eosinophil cell lysate, at least one eosinophil secondary granules basic protein or any functional fragment thereof, poly-L-arginine and any combination thereof. The composition of the invention may optionally further comprise a pharmaceutically acceptable carrier, diluent, excipient and/or additive.

According to a preferred embodiment, the eosinophil secondary granules basic protein comprised within the compositions of the invention may be selected from the group consisting of MBP (Major Basic Protein), ECP (Eosinophil Cationic Protein), EPO (Eosinophil Peroxidase) and EDN (Eosinophil Derived Neurotoxin), preferably, MBP (Major Basic Protein) or any functional fragment thereof.

According to another preferred embodiment, the eosinophil secondary granules basic protein or any functional fragment thereof comprised within the compositions of the invention, may be provided as any one of a purified recombinant protein, a fusion protein, a nucleic acid construct encoding for said protein, a host cell expressing said construct, a cell, a cell line and tissue endogeneously expressing said protein or any lysates thereof.

In a specifically preferred embodiment, the pharmaceutical composition of the invention is intended for the inhibition or the treatment of a process-associated with heparanase catalytic activity, such as for example, angiogenesis, tumor formation, tumor progression and tumor metastasis.

In another specifically preferred embodiment, the pharmaceutical composition of the invention is particularly applicable for the treatment of a pathologic disorder associated with heparanase catalytic activity, for example, a malignant proliferative disorder such as carcinoma, melanoma, leukemia, and lymphoma. In yet another specifically preferred embodiment, the pharmaceutical composition of the invention may be particularly applicable for the treatment of inflammatory disorder and autoimmune disorder.

In a second aspect, the invention relates to a method for the inhibition of heparanase glycosidase catalytic activity. The method of the invention comprises the steps of in-vivo or in-vitro contacting heparanase under suitable conditions, with an inhibitory effective amount of any one of eosinophil cell lysate, at least one eosinophil secondary granules basic protein or any functional fragments thereof, poly-L-arginine and any combination thereof, or with a composition comprising the same.

According to one embodiment of said aspect, the heparanase inhibited by the method of the invention may be in any form, for example, a purified recombinant protein, a fusion heparanase protein, a nucleic acid construct encoding heparanase, a host cell expressing said construct, a cell, a cell line and a tissue endogeneously expressing the active form of heparanase, or any lysates thereof.

The invention further provides for a method for the inhibition of heparanase glycosidase catalytic activity in a subject in need thereof. Such method comprises the steps of administering to said subject an inhibitory effective amount of any one of eosinophil cell lysate, at least one eosinophil secondary granules basic protein or any functional fragments thereof, poly-L-arginine and any combination thereof, or of a composition comprising the same.

Still further, the invention related to a method for the inhibition or the treatment of a process or a pathologic disorder associated with heparanase glycosidase catalytic activity. Such method comprises the steps of administering to a subject in need thereof a therapeutically effective amount of any one of eosinophil cell lysate, at least one eosinophil secondary granules basic protein or any functional fragments thereof, poly-L-arginine and any combination thereof, or of a composition comprising the same.

In a preferred embodiment, the eosinophil secondary granules basic protein used by the methods of the invention may be selected from the group consisting of MBP (Major Basic Protein), ECP (Eosinophil Cationic Protein), EPO (Eosinophil Peroxidase) and EDN (Eosinophil Derived Neurotoxin), preferably, MBP (Major Basic Protein) or any functional fragment thereof.

According to another embodiment, the eosinophil secondary granules basic protein or any functional fragment thereof used by the methods of the invention may be provided as a purified recombinant protein, a fusion protein, a nucleic acid construct encoding for said protein, host cell expressing said construct, or a cell, a cell line and a tissue endogeneously expressing said protein or any lysates thereof.

In another preferred embodiment, the method of the invention is intended for the treatment and inhibition of a process associated with heparanase glycosidase catalytic activity, for example, angiogenesis, tumor formation, tumor progression or tumor metastasis.

In yet another embodiment, the method of the invention is intended for the treatment of a pathologic disorder associated with heparanase glycosidase catalytic activity. A particular example for such pathologic disorder is a malignant proliferative disorder that may be according to a specific embodiment, a solid or a non-solid tumor, for example, carcinoma, melanoma, leukemia or lymphoma. Another example for a pathologic disorder may be an inflammatory disorder, a kidney disorder or autoimmune disorder.

It should be specifically appreciated that the method of the invention may be applicable for example, for example, the treatment of melanoma and metastatic melanoma.

According to a third aspect, the invention relates to the use of any one of eosinophil cell lysate, at least one eosinophil secondary granules basic protein or any functional fragment thereof, poly-L-arginine and any combination thereof, for the inhibition of heparanase glycosidase catalytic activity.

The invention further relates to the use of any one of eosinophil cell lysate, at least one eosinophil secondary granules basic protein or any functional fragment thereof, poly-L-arginine and any combination thereof, in the preparation of a composition for the inhibition of heparanase glycosidase catalytic activity.

Still further, the invention relates to the use of any one of eosinophil cell lysate, at least one eosinophil secondary granules basic protein or any functional fragment thereof, poly-L-arginine and any combination thereof, in the preparation of a pharmaceutical composition for the treatment or the inhibition of a process or a pathologic disorder associated with heparanase glycosidase catalytic activity. Such composition optionally further comprising a pharmaceutically acceptable carrier, diluent, excipient and/or additive.

According to a specifically preferred embodiment, the eosinophil secondary granules basic protein used by the invention may be selected from the group consisting of MBP (Major Basic Protein), ECP (Eosinophil Cationic Protein), EPO (Eosinophil Peroxidase), and EDN (Eosinophil Derived Neurotoxin), preferably, MBP (Major Basic Protein) or any functional fragment thereof.

The eosinophil secondary granules basic protein or any functional fragment thereof used by the invention, may be provided as any one of a purified recombinant protein, a fusion protein, a nucleic acid construct encoding for said protein, a host cell expressing said construct, a cell, a cell line and a tissue endogeneously expressing said protein or any lysates thereof.

According to a specifically preferred embodiment, the use according to the invention is particularly applicable for the preparation of a pharmaceutical composition for the inhibition or the treatment of a process associated with heparanase glycosidase catalytic activity, which may be angiogenesis, tumor formation, tumor progression or tumor metastasis.

In yet another embodiment, the use according to the invention is intended for the preparation of pharmaceutical compositions for the treatment of a pathologic disorder associated with heparanase glycosidase catalytic activity, such as a malignant proliferative disorder, an inflammatory disorder or an autoimmune disorder.

### BRIEF DESCRIPTION OF THE FIGURES

The invention will be further described by the hand of the proceeding Figures.

FIG. 1A-1C Heparanase expression in human eosinophils

FIG. 1A. RT-PCR. Lane 1:300 bp band amplified from the mRNA of human eosinophils. Lane 2: Human heparanase cDNA. Control PCR (no RT) was negative (not shown).

FIG. 1B. Western blot. Lane 1: Eosinophil lysates, incubated with HS-conjugated beads, electrophoresed and blotted with anti-heparanase antibodies. Lane 2: Recombinant human heparanase, consisting mostly of the 65 kDa latent enzyme.

FIG. 1C. Immunostaining. Eosinophils were incubated with anti-heparanase antibodies and stained with peroxidase-conjugated secondary antibodies (Red-brown).

FIG. 2A-2C Heparanase and MBP Co-Localization in Eosinophils: Confocal Microscopy

FIG. 2A. Staining of MBP (red).

FIG. 2B. Staining of heparanase (green).

FIG. 2C. Double immunostaining of heparanase and MBP. Partial co-localization of heparanase and MBP within the cells (yellow).

Inset: Western blot. Lane 1: Eosinophil heparanase co-immunoprecipitated with anti-MBP and was detected using anti-heparanase antibodies. Lane 2: Human foreskin fibroblasts immunoprecipitated, as in Lane 1. Lane 3: Recombinant human heparanase, consisting primarily of the 50 kDa enzyme.

FIG. 3 Lack of heparanase activity in resting human eosinophils

Lysates of human eosinophils (∘) and neutrophils (♦) (1×10⁶/ml) were incubated with intact biosynthetically ³⁵[S]-labeled ECM. Labeled degradation fragments released into the incubation medium were analyzed by gel filtration on Sepharose 6B. Abbreviations: Frac. (fraction) Sulf. Lab. Mat. Cpm (sulfate labeled material, counts per minute).

FIG. 4A-4B Lack of heparanase activity in activated human eosinophils

FIG. 4A. EPO release in the supernatants of eosinophils pre-incubated with medium in the presence (▪) or absence (□) of GM-CSF followed by C5a stimulation (absorbance, 490 nM) [Kuhry J G. et al. Agents Actions 16:109-12 (1985)].

FIG. 4B. Heparanase activity (was evaluated as described for FIG. 3) of supernatants of GM-CSF/C5a unactivated (∘) or activated (●) eosinophils. The ECM was also incubated with of recombinant heparanase (□). Abbreviations: Frac. (fraction) Sulf. Lab. Mat. Cpm (sulfate labeled material, counts per minute), Abs. (absorbance).

FIG. 5A-5C Inhibition of Heparanase Activity by Eosinophils and MBP

Recombinant 50 kDa human heparanase (10 ng/ml) was incubated with ³⁵[S]-ECM in the absence (♦) or presence of eosinophil lysates (∘), or human foreskin fibroblast lysates (∘) (FIG. 5A); in the absence (♦), or presence of 2×10⁻⁷ M (∘), or 0.8×10⁻⁷ M (∘) purified MBP (FIG. 5B); in the absence (♦) or presence of MBP (⋄), EPO (∘), or ECP (Δ), each at a concentration of 4×10⁻⁷ M (FIG. 5C) (see legend FIG. 3). Abbreviations: Frac. (fraction) Sulf. Lab. Mat. Cpm (sulfate labeled material, counts per minute).

FIG. 6 Heparanase activity in the peritoneal lavage of mice with allergic peritonitis

TNF-KO (∘) and control WT mice (♦) were sensitized and challenged with OVA (ovalbumin) to induce allergic peritonitis. Three days after challenge, peritoneal lavage was performed and the supernatants assessed for heparanase activity (see legend 3). Abbreviations: Frac. (fraction) Sulf. Lab. Mat. Cpm (sulfate labeled material, counts per minute).

FIG. 7A-7B Inhibition of melanoma lung metastasis by MBP

B-16 melanoma cells, incubated for 15 min in the presence of purified MBP or saline (control), and then injected to the tail vein of C57BL6 mice. Sixteen days later, the mice were scarified and the number of surface metastatic colonies was counted in their lungs.

FIG. 7A upper panel shows lungs from mice inoculated with cells treated with saline (control), lower panel shows lungs from mice inoculated with MBP treated cells.

FIG. 7B Schematic presentation of the number of metastatic lesions in the lungs of the mice inoculated with saline (31±8 colonies/mouse) vs. MBP (2±0.3 colonies/mouse) treated cells. Mean±se.

### DETAILED DESCRIPTION OF PREFERRED EMBODIMENTS OF THE INVENTION

A number of methods of the art of molecular biology are not detailed herein, as they are well known to the person of skill in the art. Such methods include site-directed mutagenesis, PCR cloning, expression of cDNAs, analysis of recombinant proteins or peptides, transformation of bacterial and yeast cells, transfection of mammalian cells, and the like. Textbooks describing such methods are e.g., Sambrook et al., Molecular Cloning A Laboratory Manual, Cold Spring Harbor Laboratory; ISBN: 0879693096,1989, Current Protocols in Molecular Biology, by F. M. Ausubel, ISBN: 047150338X, John Wiley & Sons, Inc. 1988, and Short Protocols in Molecular Biology, by F. M. Ausubel et al., (eds.) 3rd ed. John Wiley & Sons; ISBN: 0471137812, 1995. These publications are incorporated herein in their entirety by reference. Furthermore, a number of immunological techniques are not in each instance described herein in detail, as they are well known to the person of skill in the art. See e.g., Current Protocols in Immunology, Coligan et al., (eds), John Wiley & Sons. Inc., New York, N.Y.

The present invention demonstrates for the first time that eosinophil-derived MBP efficiently inhibits heparanase activity. This inhibitory effect may be relevant in vivo, since MBP is released from eosinophils in several diseases in which heparanase is also involved. For example, eosinophils, besides being effector cells of allergic and parasitic reactions, have different roles in fibrosis, cancer and autoimmune diseases [Gleich (2000) ibid.; Levi-Schaffer (1999) ibid.; Samoszuk (1997) ibid.], where heparanase could degrade the ECM scaffold and hence facilitate the penetration of eosinophils into tissues.

In the present application, the inventors found that eosinophils, as other inflammatory cells [Vlodavsky (1992) ibid.; Matzner (1985) ibid.] produce heparanase. However, the inventors could not detect any heparanase enzymatic activity in resting eosinophils or in eosinophils activated with various agonists [Simon (2000) ibid.; Temkin (2002) ibid.; Hoenstein (2001) ibid.], including GM-CSF/C5a, one of the most potent eosinophil degranulators [Simon (2000) ibid.]. Likewise, co-culture of eosinophils with cells that did not express intrinsic heparanase but do interact with eosinophils and modulate their functional activity [Rothenberg, M. E. et al., Science 237:645-7 (1987); Hallsworth, M. P. et al., Am. J. Respir. Cell Mol. Biol. 19:910-9 (1998); Solomon, A. et al., Invest. Ophthalmol. Vis. Sci. 41:1038-44 (2000)], or that process and activate exogenously added latent heparanase [Nadav, L. et al., J. Cell Sci. 115:2179-87 (2002)] did not result in heparanase activity.

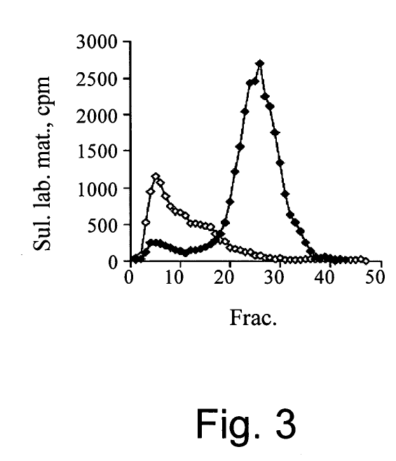

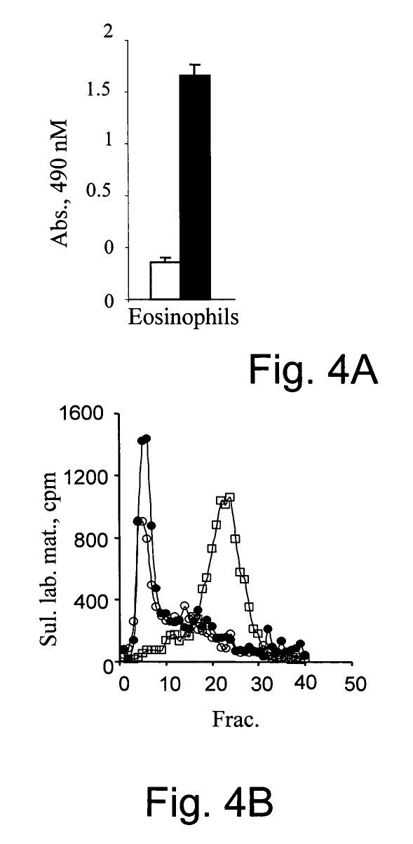

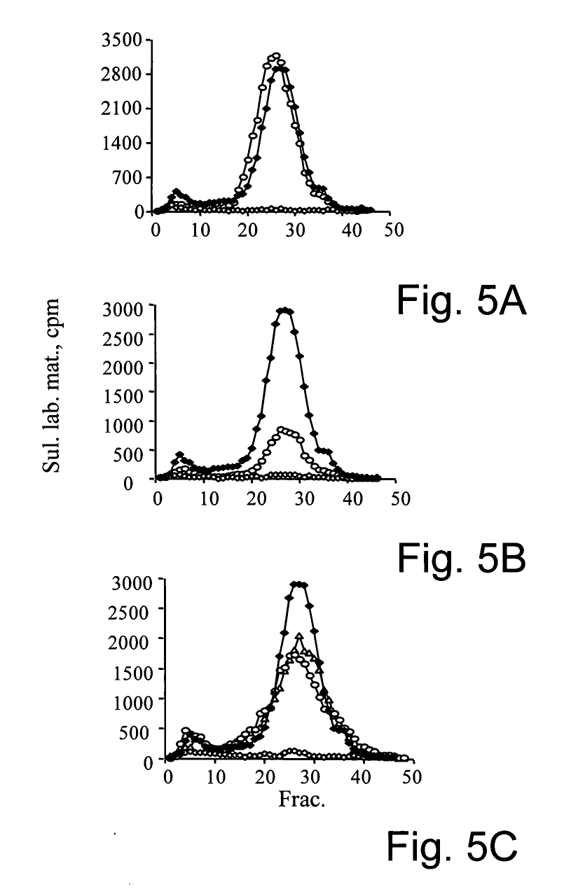

The inventors therefore hypothesized that eosinophils might contain a substance that inhibits heparanase. As shown by FIGS. 3 and 4, eosinophil lysates, as well as a highly purified preparation of MBP (FIG. 5), inhibited heparanase activity in a dose-dependent manner, reaching 100% at a concentration of 2×10⁻⁷ M. Complete inhibition was observed even at an estimated lower excess (˜2.5 folds) of cellular MBP over heparanase.

Thus, in a first aspect, present invention relates to a composition for the inhibition of heparanase glycosidase catalytic activity. Such composition comprises as an active ingredient any one of eosinophil cell lysate, at least one eosinophil secondary granules basic protein or any functional fragment thereof, poly-L-arginine and any combination thereof. It should be appreciated that these eosinophils secondary granules proteins may also include proteins associated with crystalloid granules. The composition of the invention may optionally further comprise a pharmaceutically acceptable carrier, diluent, excipient and/or additive.

According to a one preferred embodiment, the eosinophil secondary granules basic protein may be selected from the group consisting of MBP (Major Basic Protein), ECP (Eosinophil Cationic Protein), EPO (Eosinophil Peroxidase) and EDN (Eosinophil Derived Neurotoxin).

While natural inhibitors of matrix degrading proteases such as tissue inhibitors of matrix metalloproteinases and plasminogen activator inhibitors are well characterized [Stetler-Stevenson, W. G. and Yu, A. E. Semin. Cancer Biol. 11:143-52 (2001)], MBP is the first naturally occurring protein that efficiently inhibits heparanase enzymatic activity. Structurally, MBP is characterized by 14% arginine residues (17/117 amino acids) and only a single acidic residue, aspartic acid, yielding a calculated p/value of 11.4 [Gleich, G. J. J. et al., Clin. Invest. 57:633-40 (1976)]. Notably, both ECP and EPO, that were found to exert a partial inhibitory activity towards heparanase, also contain arginine [Mallorqui-Fernandez, G. et al., J. Mol. Biol. 300:1297-307 (2000); Carlson, M. G. et al., J. Immunol. 134:1875-9 (1985)], although to a much lower extent than MBP. MBP also contains nine cysteines, four of which form two disulfide bonds that are homologous to the two disulfide bonds conserved in the carbohydrate recognition domain of C-type lectins [Thomas (2001) ibid.]. It is therefore conceivable that these polycationic proteins may interact with the polyanionic HS substrate and hence protect it from cleavage and/or diminish its accessibility to heparanase. In preliminary experiments, the inventors found that heparanase pre-incubated with MBP co-precipitated when anti-MBP antibodies were added, indicating that a direct interaction between the two proteins is feasible. Other basic compounds such as compound 48/80 and myelin basic protein [Kuhry (1985) ibid.; Chekhonin (2000) ibid.], only partially inhibit heparanase activity. Poly-L-arginine at very high concentrations caused an almost complete inhibition. This, together with the previous results, strongly suggests a specific interaction of MBP with the heparanase enzyme, resulting in inhibition of its enzymatic activity.

In a murine model of allergic peritonitis [Temkin (2003) ibid.] the inventors found that peritoneal cavity heparanase activity, probably originated from eosinophils and other inflammatory cells, was inversely related to the eosinophil number. This would indicate that heparanase can be inhibited by the “excess” of MBP released by activated eosinophils.

Therefore, according to a specifically preferred embodiment, the invention relates to a composition for the inhibition of heparanase glycosidase catalytic activity, comprising as an active ingredient an inhibitory effective amount of MBP (Major Basic Protein) or any functional fragment thereof. Such composition may optionally further comprises a pharmaceutically acceptable carrier, diluent, excipient and/or additive.

As used herein in the specification and in the claims section below, the phrase “heparanase glycosidase catalytic activity”, “heparanase catalytic activity” or its equivalent “heparanase activity” refers to an animal endoglycosidase hydrolyzing activity which is specific for heparin or heparan sulfate proteoglycan substrates, as opposed to the activity of bacterial enzymes (heparinase I, II and III) which degrade heparin or heparan sulfate by means of 1-elimination. Heparanase activity which is inhibited or neutralized according to the present invention can be of either recombinant or natural heparanase. Such activity is disclosed, for example, in U.S. Pat. No. 6,177,545 and U.S. Pat. No. 6,190,875, which are incorporated by reference as if fully set forth herein.

As used herein in the specification and in the claims section below, the term “inhibit” and its derivatives refers to suppress or restrain from free expression of activity. According to a preferred embodiment of the present invention at least about 60-70%, preferably, at least about, 70-80%, more preferably, at least about 80-90% most preferably, at least about 90-100% of the heparanase activity is abolished by MBP, as shown by the invention hereinafter.

By “functional fragments” is meant “fragments”, “variants”, “analogs” or “derivatives” of the molecule. A “fragment” of a molecule, such as any of the amino acid sequence of the MBP protein used by the present invention is meant to refer to any amino acid subset of the molecule. A “variant” of such molecule is meant to refer to a naturally occurring molecule substantially similar to either the entire molecule or a fragment thereof. An “analog” of a molecule is a homologous molecule from the same species or from different species. By “functional” is meant having same biological function, for example, having identical ability to inhibit heparanase catalytic activity.

It should be appreciated that the eosinophil secondary granules basic protein or any functional fragment thereof comprised within the composition of the invention may be provided as any one of a purified recombinant protein, a fusion protein, a nucleic acid construct encoding such protein, a host cell expressing this construct, a cell, a cell line and tissue endogeneously expressing said eosinophil secondary granules basic protein or any lysates thereof.

As indicated above, the eosinophil secondary granules basic protein or compositions thereof may be provided as nucleic acid constructs. As used herein, the term “nucleic acid” refers to polynucleotides such as deoxyribonucleic acid (DNA), and, where appropriate, ribonucleic acid (RNA). The terms should also be understood to include, as equivalents, analogs of either RNA or DNA made from nucleotide analogs, and, as applicable to the embodiment being described, single-stranded and double-stranded polynucleotides. “Constructs”, as used herein, encompass vectors such as plasmids, viruses, bacteriophage, integratable DNA fragments, and other vehicles, which enable the integration of DNA fragments into the genome of the host. Expression vectors or constructs are typically self-replicating DNA or RNA constructs containing the desired gene or its fragments, and operably linked genetic control elements that are recognized in a suitable host cell and effect expression of the desired genes. These control elements are capable of effecting expression within a suitable host. Generally, the genetic control elements can include a prokaryotic promoter system or an eukaryotic promoter expression control system. This typically includes a transcriptional promoter, an optional operator to control the onset of transcription, transcription enhancers to elevate the level of RNA expression, a sequence that encodes a suitable ribosome binding site, RNA splice junctions, sequences that terminate transcription and translation and so forth. Expression vectors usually contain an origin of replication that allows the vector to replicate independently of the host cell.

A vector may additionally include appropriate restriction sites, antibiotic resistance or other markers for selection of vector containing cells. Plasmids are the most commonly used form of vector but other forms of vectors which serves an equivalent function and which are, or become, known in the art are suitable for use herein. See, e.g., Pouwels et al., Cloning Vectors: a Laboratory Manual (1985 and supplements), Elsevier, N.Y.; and Rodriquez, et al., (eds.) Vectors: a Survey of Molecular Cloning Vectors and their Uses, Buttersworth, Boston, Mass. (1988), which are incorporated herein by reference.

Also, a specific embodiment of the invention relates to a host cell transformed with a construct expressing said eosinophil secondary basic protein. Suitable host cells include prokaryotes, lower eukaryotes, and higher eukaryotes. Prokaryotes include gram negative and gram positive organisms, e.g., E. coli and B. subtilis. Lower eukaryotes include yeast, S. cerevisiae and Pichia, and species of the genus Dictyostelium. Higher eukaryotes include established tissue culture cell lines from animal cells, both of non-mammalian origin, e.g., insect cells and birds, and of mammalian origin, e.g., human and other primate, and of rodent origin.

“Cells”, “host cells” or “recombinant cells” are terms used interchangeably herein. It is understood that such terms refer not only to the particular subject cells but to the progeny or potential progeny of such a cell. Because certain modification may occur in succeeding generation due to either mutation or environmental influences, such progeny may not, in fact, be identical to the parent cell, but are still included within the scope of the term as used herein.

“Host cell” as used herein refers to cells which can be recombinantly transformed with vectors constructed using recombinant DNA techniques. A drug resistance or other selectable marker is intended in part to facilitate the selection of the transformants. Additionally, the presence of a selectable marker, such as drug resistance marker may be of use in keeping contaminating microorganisms from multiplying in the culture medium. Such a pure culture of the transformed host cell would be obtained by culturing the cells under conditions which require the induced phenotype for survival.

As used herein, the term “transfection” means the introduction of a nucleic acid, e.g., an expression vector, into a recipient cells by nucleic acid-mediated gene transfer. “Transformation”, as used herein, refers to a process in which a cell's genotype is changed as a result of the cellular uptake of exogenous DNA or RNA.

The invention further provides a pharmaceutical composition for the treatment or the inhibition of a process or a pathologic disorder associated with heparanase catalytic activity. Such composition comprises as an active ingredient any one of eosinophil cell lysate, at least one eosinophil secondary granules basic protein or any functional fragment thereof, poly-L-arginine and any combination thereof, in an amount sufficient for the inhibition of heparanase glycosidase catalytic activity. The pharmaceutical composition of the invention may optionally further comprise a pharmaceutically acceptable carrier, diluent, excipient and/or additive.

According to a preferred embodiment, the eosinophil secondary granules basic protein comprised within the pharmaceutical composition of the invention may be selected from the group consisting of MBP (Major Basic Protein), ECP (Eosinophil Cationic Protein), EPO (Eosinophil Peroxidase) and EDN (Eosinophil Derived Neurotoxin).

The heparanase inhibitors of the present invention can be used for the treatment of diseases and disorders caused by or associated with heparanase catalytic activity. As used herein in the specification and in the claims section below, the phrase “associated with heparanase catalytic activity” refers to conditions which at least partly depend on the catalytic activity of heparanase. It is being understood that the catalytic activity of heparanase under many such conditions can be normal, yet inhibition thereof in such conditions will result in improvement of the affected individual.

It should be further noted that disorders or the condition can be related to altered function of a HSPG associated biological effector molecule, such as, but not limited to, growth factors, chemokines, cytokines and degradative enzymes. The condition can be, or involve, angiogenesis, tumor cell proliferation, invasion of circulating tumor cells, metastases, inflammatory disorders, autoimmune conditions and/or a kidney disorder.

Given the potential tissue damage that could result from cleavage of HS, tight regulation of heparanase expression and activity is essential. An attractive regulatory target is the apparently membrane bound, not yet identified protease that converts the heparanase from a latent 65 kDa protein into an active 50 kDa form [Vlodavsky (2001) ibid.; Fairbanks (1999) ibid.]. Regulation of heparanase promoter activity is being investigated, emphasizing the inhibitory effect of methylation and stimulation by estrogen [Shteper, P. J. et al., Oncogene 22:7737-7749 (2003)]. In vivo models of cancer metastasis and angiogenesis, have shown that the potent pro-angiogenic and pro-metastatic properties of heparanase are tightly regulated by its cellular localization and secretion [Goldshmidt, O. et al., Exp. Cell. Res. 281:50-62 (2002)]. Thus, cell surface binding and routing of heparanase into late endosomes appear to control its activation, clearance, and storage within the cells [Nadav (2002) ibid.; Goldshmidt, O. et al., Proc. Natl. Acad. Sci. USA 99:10031-6 (2002)]. The results described by the present application suggests that inhibition of heparanase by MBP may provide a protective feedback mechanism that can halt tissue damage in response to excess heparanase secreted at sites of massive inflammation [Vlodavsky (1992) ibid.; Mollinedo, F. et al., Biochem J 327:917-23 (1997)].

The heparanase enzyme has been identified in platelets and neutrophils, and is readily released in an active form upon their degranulation. In contrast, this enzyme may be released from activated eosinophils as a complex with MBP and possible other granular mediators, but in an inactive form due to the inhibitory effect of MBP. The novel anti-heparanase activity of MBP contrasts sharply with the previously established involvement of eosinophils and MBP in tissue damage that appears to be important in allergy, parasitic diseases and certain cancers.

MPB is the first natural protein inhibitor of heparanase activity to be identified. In view of the increasing significance of heparanase as a target for anti-cancer drug development, MBP and related compounds are being evaluated in experimental models of cancer metastasis and angiogenesis as candidates for potential application in cancer treatment.

Therefore, according to a specifically preferred embodiment, the invention provides for a pharmaceutical composition, for the inhibition or the treatment of a process or a pathologic disorder associated with heparanase glycosidase catalytic activity. This particular composition comprises as an active ingredient a MBP (Major Basic Protein) or any functional fragment thereof in an amount sufficient for the inhibition of heparanase catalytic activity.

According to another preferred embodiment, the eosinophil secondary granules basic protein or any functional fragment thereof comprised within composition of the invention, may be provided as any one of a purified recombinant protein, a fusion protein, a nucleic acid construct encoding for said protein, a host cell expressing said construct, a cell, a cell line and tissue endogeneously expressing said protein or any lysates thereof.

In a specifically preferred embodiment, the pharmaceutical composition of the invention is intended for the inhibition or the treatment of a process-associated with heparanase catalytic activity, such as for example, angiogenesis, tumor formation, tumor progression and tumor metastasis.

Involvement in tumor angiogenesis of heparanase has been correlated with the ability to release bFGF (FGF-2) and other growth factors from its storage within the ECM (extracellular matrix). These growth factors provide a mechanism for induction of neovascularization in normal and pathological situations.

Heparanase may thus facilitate not only tumor cell invasion and metastasis but also tumor angiogenesis, both critical steps in tumor progression.

It is to be therefore understood that the compositions of the invention are useful for treating or inhibiting tumors at all stages, namely tumor formation, primary tumors, tumor progression or tumor metastasis.

Thus, in one embodiment of the present invention, the compositions of the invention can be used for inhibition of angiogenesis, and are thus useful for the treatment of diseases and disorders associated with angiogenesis or neovascularization such as, but not limited to, tumor angiogenesis, opthalmologic disorders such as diabetic retinopathy and macular degeneration, particularly age-related macular degeneration, and reperfusion of gastric ulcer.

The eosinophil secondary granules basic protein as well as the compositions of the invention described herein, were characterized in the present application as exhibiting a high power of inhibiting heparanase, and are therefore useful as active ingredients for the preparation of medicaments for the treatment of pathologies gaining benefit from the inhibition of the heparanase, pathologies based on abnormal angiogenesis, and particularly for the treatment of metastases, for example, metastatic lung carcinoma as shown by Example 5.

In another specifically preferred embodiment, the pharmaceutical composition of the invention is particularly applicable for the treatment of a pathologic disorder associated with heparanase catalytic activity, for example, a malignant proliferative disorder.

As used herein to describe the present invention, “malignant proliferative disorder” “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the composition as well as the methods of the present invention may be used in the treatment of non-solid and solid tumors, for example, carcinoma, melanoma, leukemia, and lymphoma.

As shown by Example 5, the pharmaceutical compositions of the invention are applicable for example, for the treatment of melanoma, and metastasis thereof, specifically, in lungs.

The term melanoma includes, but is not limited to, melanoma, metastatic melanoma, melanoma derived from either melanocytes or melanocyte-related nevus cells, melanocarcinoma, melanoepithelioma, melanosarcoma, melanoma in situ, superficial spreading melanoma, nodular melanoma, lentigo maligna melanoma, acral lentiginoous melanoma, invasive melanoma or familial atypical mole and melanoma (FAM-M) syndrome. Such melanomas may be caused by chromosomal abnormalities, degenerative growth and developmental disorders, mitogenic agents, ultraviolet radiation (UV), viral infections, inappropriate tissue gene expression, alterations in gene expression, or carcinogenic agents. The aforementioned melanomas can be treated by the method and the composition described in the present invention.

Furthermore, according to a preferred embodiment, the eosinophil secondary granules basic protein or a composition comprising the same, can be used for the treatment or inhibition of non-solid cancers, e.g. hematopoietic malignancies such as all types of leukemia, e.g. acute lymphocytic leukemia (ALL), acute myelogenous leukemia (AML), chronic lymphocytic leukemia (CLL), chronic myelogenous leukemia (CML), myelodysplastic syndrome (MDS), mast cell leukemia, hairy cell leukemia, Hodgkin's disease, non-Hodgkin's lymphomas, Burkitt's lymphoma and multiple myeloma, as well as for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma and Kaposi's sarcoma.

The eosinophil secondary granules basic protein, and particularly MBP or any compositions thereof may be also useful for inhibiting or treating other cell proliferative diseases or disorders such as psoriasis, hypertrophic scars, fibrosis resulting from surgical intervention, acne and sclerosis/scleroderma, and for inhibition or treatment of other diseases or disorders such as polyps, multiple exostosis, hereditary exostosis, retrolental fibroplasia, hemangioma, idiopatic fibrotic diseases and arteriovenous malformation.

In a further embodiment, the compositions of the invention may be useful for treatment of or amelioration of inflammatory symptoms in any disease, condition or disorder where immune and/or inflammation suppression is beneficial such as, but not limited to, treatment of or amelioration of inflammatory symptoms in the joints, musculoskeletal and connective tissue disorders, or of inflammatory symptoms associated with hypersensitivity, allergic reactions, asthma, atherosclerosis, otitis and other otorhinolaryngological diseases, dermatitis and other skin diseases, posterior and anterior uveitis, conjunctivitis, optic neuritis, scleritis and other immune and/or inflammatory ophthalmic diseases.

In another preferred embodiment, the compositions of the invention are useful for treatment of or amelioration of an autoimmune disease such as, but not limited to, Eaton-Lambert syndrome, Goodpasture's syndrome, Greave's disease, Guillain-Barr syndrome, autoimmune hemolytic anemia (AI HA), hepatitis, insulin-dependent diabetes mellitus (IDDM), systemic lupus erythematosus (SLE), multiple sclerosis (MS), myasthenia gravis, plexus disorders e.g. acute brachial neuritis, polyglandular deficiency syndrome, primary biliary cirrhosis, rheumatoid arthritis, scleroderma, thrombocytopenia, thyroiditis e.g. Hashimoto's disease, Sjbgren's syndrome, allergic purpura, psoriasis, mixed connective tissue disease, polymyositis, dermatomyositis, vasculitis, polyarteritis nodosa, polymyalgia rheumatica, Wegener's granulomatosis, Reiter's syndrome, Behget's syndrome, ankylosing spondylitis, pemphigus, bullous pernphigoid, dennatitis herpetiformis, insulin dependent diabetes, inflammatory bowel disease, ulcerative colitis and Crohn's disease.

Pharmaceutical compositions for use in accordance with the present invention may be formulated in conventional manner using one or more physiologically acceptable carriers or excipients. The carrier(s) must be acceptable in the sense that it is compatible with the other ingredients of the composition and it is not deleterious to the recipient thereof.

The term “carrier” refers to a diluent, adjuvant, excipient, or any other suitable vehicle. Such pharmaceutical carriers can be sterile liquids such as water and oils. “Pharmaceutically acceptable carrier” includes any and all solvents, dispersion media, coatings, antibacterial and antifungal agents and the like. The use of such media and agents for pharmaceutical active substances is well known in the art. Except as any conventional media or agent is incompatible with the active ingredient, its use in the therapeutic composition is contemplated.

In a second aspect, the invention relates to a method for the inhibition of heparanase glycosidase catalytic activity. The method of the invention comprises the steps of in-vivo or in-vitro contacting heparanase under suitable conditions for example as demonstrated by the Examples, with an inhibitory effective amount of any one of eosinophil cell lysate, at least one eosinophil secondary granules basic protein or any functional fragments thereof, poly-L-arginine and any combination thereof, or with a composition comprising the same.

According to one embodiment of said aspect, the heparanase inhibited by the method of the invention may be in any form, for example, inhibition of heparanase in vitro by the method of the invention, may be performed by using heparanase as a purified recombinant protein, a fusion heparanase protein, a nucleic acid construct encoding heparanase, or alternatively, in tissue culture of a host cell expressing said construct, a cell, or cell line endogeneously expressing the active form of heparanase, or lysates thereof. A method for the inhibition of heparanase in vivo may be performed in a tissue endogeneously expressing the active form of heparanase.

The invention further provides for a method for the inhibition of heparanase glycosidase catalytic activity in a subject in need thereof. Such method comprises the steps of administering to said subject an inhibitory effective amount of any one of eosinophil cell lysate, at least one eosinophil secondary granules basic protein or any functional fragments thereof, poly-L-arginine and any combination thereof, or of a composition comprising the same.

In a preferred embodiment, the eosinophil secondary granules basic protein used by the methods of the invention may be selected from the group consisting of MBP (Major Basic Protein), ECP (Eosinophil Cationic Protein), EPO (Eosinophil Peroxidase) and EDN (Eosinophil Derived Neurotoxin).

In a specifically preferred embodiment, the eosinophil secondary granules basic protein used by the methods of the invention is MBP (Major Basic Protein) or any functional fragment thereof.

According to another embodiment, the eosinophil secondary granules basic protein or any functional fragment thereof used by the method of the invention may be provided as a purified recombinant protein, a fusion protein, a nucleic acid construct encoding for said protein, host cell expressing said construct, or a cell, a cell line and a tissue endogeneously expressing said protein or any lysates thereof.

Still further, the invention related to a method for the inhibition or the treatment of a process or a pathologic disorder associated with heparanase glycosidase catalytic activity. Such method comprises the steps of administering to a subject in need thereof a therapeutically effective amount of any one of eosinophil cell lysate, at least one eosinophil secondary granules basic protein or any functional fragments thereof, poly-L-arginine and any combination thereof, or of a composition comprising the same.

As used herein in the specification and in the claims section below, the term “treat” or treating and their derivatives includes substantially inhibiting, slowing or reversing the progression of a condition, substantially ameliorating clinical symptoms of a condition or substantially preventing the appearance of clinical symptoms of a condition.

More specifically, the eosinophil secondary granules basic protein used by the method of the invention may be selected from the group consisting of MBP (Major Basic Protein), ECP (Eosinophil Cationic Protein), EPO (Eosinophil Peroxidase) and EDN (Eosinophil Derived Neurotoxin).

Preferably, MBP (Major Basic Protein) or any functional fragment thereof, may be used for the method of the invention.

According to a preferred embodiment, the eosinophil secondary granules basic protein or any functional fragment thereof used by the method of the invention may be provided as any one of a purified recombinant protein, a fusion protein, a nucleic acid construct encoding for said protein, a host cell expressing said construct, a cell, a cell line and a tissue endogeneously expressing said protein or any lysates thereof.

In another preferred embodiment, the method of the invention is intended for the treatment and inhibition of a process associated with heparanase glycosidase catalytic activity, for example, angiogenesis, tumor formation, tumor progression or tumor metastasis.

In yet another embodiment, the method of the invention is intended for the treatment of a pathologic disorder associated with heparanase glycosidase catalytic activity. A particular example for such pathologic disorder is a malignant proliferative disorder that may be according to a specific embodiment, a solid or a non-solid tumor, for example, carcinoma, melanoma, leukemia or lymphoma, particularly, melanoma.

Still further, the method of the invention is intended for the treatment of a pathologic disorder, such as inflammatory disorder, a kidney disorder or autoimmune disorder.

The pharmaceutical compositions of the invention may be administered systemically, for example by parenteral, e.g. intravenous, intraperitoneal or intramuscular injection. In another example, the pharmaceutical composition can be introduced to a site by any suitable route including intravenous, subcutaneous, transcutaneous, topical, intramuscular, intraarticular, subconjunctival, or mucosal, e.g. oral, intranasal, or intraocular administration.

Local administration to the area in need of treatment may be achieved by, for example, local infusion during surgery, topical application, direct injection into the inflamed joint, directly onto the eye, etc.

For oral administration, the pharmaceutical preparation may be in liquid form, for example, solutions, syrups or suspensions, or in solid form as tablets, capsules and the like. For administration by inhalation, the compositions are conveniently delivered in the form of drops or aerosol sprays. For administration by injection, the formulations may be presented in unit dosage form, e.g. in ampoules or in multidose containers with an added preservative.

The pharmaceutical forms suitable for injection use include sterile aqueous solutions or dispersions and sterile powders for the extemporaneous preparation of sterile injectable solutions or dispersions. In all cases the form must be sterile and must be fluid to the extent that easy syringeability exists. It must be stable under the conditions of manufacture and storage and must be preserved against the contaminating action of microorganisms, such as bacterium and fungi.

The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.
n the case of sterile powders for the preparation of the sterile injectable solutions, the preferred method of preparation are vacuum-drying and freeze drying techniques which yield a powder of the active ingredient plus any additional desired ingredient from a previously sterile-filtered solution thereof.

The compositions of the invention can also be delivered in a vesicle, for example, in liposomes. In another embodiment, the compositions can be delivered in a controlled release system.

The amount of the therapeutic or pharmaceutical composition of the invention which is effective in the treatment of a particular disease, condition or disorder will depend on the nature of the disease, condition or disorder and can be determined by standard clinical techniques. In addition, in vitro assays as well in vivo experiments may optionally be employed to help identify optimal dosage ranges. The precise dose to be employed in the formulation will also depend on the route of administration, and the seriousness of the disease, condition or disorder, and should be decided according to the judgment of the practitioner and each patient's circumstances. Effective doses may be extrapolated from dose-response curves derived from in vitro or animal model test systems.

As used herein, “effective amount” means an amount necessary to achieve a selected result. For example, an effective amount of the composition of the invention useful for inhibition of heparanase activity and thereby for the treatment of said pathology.

It should be appreciated that the method of the invention is intended for treating a mammalian subject, preferably, a human. Therefore, by “patient” or “subject in need” is meant any mammal for which such therapy is desired, including human bovine, equine, canine, and feline subjects, preferably, human patient.

In a third aspect, the invention relates to the use of any one of eosinophil cell lysate, at least one eosinophil secondary granules basic protein or any functional fragment thereof, poly-L-arginine and any combination thereof, for the inhibition of heparanase glycosidase catalytic activity.

The invention further relates to the use of any one of eosinophil cell lysate, at least one eosinophil secondary granules basic protein or any functional fragment thereof, poly-L-arginine and any combination thereof, in the preparation of a composition for the inhibition of heparanase glycosidase catalytic activity.

According to a specifically preferred embodiment, the eosinophil secondary granules basic protein used by the invention may be selected from the group consisting of MBP (Major Basic Protein), ECP (Eosinophil Cationic Protein), EPO (Eosinophil Peroxidase) and EDN (Eosinophil Derived Neurotoxin).

According to a specifically preferred embodiment, the invention relates to the use of an inhibitory effective amount of MBP (Major Basic Protein) or any functional fragment thereof in the preparation of a composition for the inhibition of heparanase glycosidase catalytic activity.

The eosinophil secondary granules basic protein or any functional fragment thereof used by the invention, may be provided as any one of a purified recombinant protein, a fusion protein, a nucleic acid construct encoding for said protein, a host cell expressing said construct, a cell, a cell line and a tissue endogeneously expressing said protein or any lysates thereof.

Still further, the invention relates to the use of any one of eosinophil cell lysate, at least one eosinophil secondary granules basic protein or any functional fragment thereof, poly-L-arginine and any combination thereof, in the preparation of a pharmaceutical composition for the treatment or the inhibition of a process or a pathologic disorder associated with heparanase glycosidase catalytic activity. Such composition optionally further comprising a pharmaceutically acceptable carrier, diluent, excipient and/or additive.

In a specific embodiment, the eosinophil secondary granules basic protein used by the invention for preparing a pharmaceutical composition, may be selected from the group consisting of MBP (Major Basic Protein), ECP (Eosinophil Cationic Protein), EPO (Eosinophil Peroxidase) and EDN (Eosinophil Derived Neurotoxin). Preferably, the invention relates to the use of MBP (Major Basic Protein) or any functional fragment thereof, in an amount sufficient for the inhibition of heparanase catalytic activity, in the preparation of a pharmaceutical composition for the inhibition or the treatment of a process or a pathologic disorder associated with heparanase catalytic activity.

In a specific embodiment, the eosinophil secondary granules basic protein or any functional fragment thereof used by the invention may be provided as any one of a purified recombinant protein, a fusion protein, a nucleic acid construct encoding for said protein, a host cell expressing said construct, a cell, a cell line and a tissue endogeneously expressing said protein or any lysates thereof.

According to a specifically preferred embodiment, the use according to the invention is particularly applicable for the preparation of a pharmaceutical composition for the inhibition or the treatment of a process associated with heparanase glycosidase catalytic activity, which may be angiogenesis, tumor formation, tumor progression or tumor metastasis.

In yet another embodiment, the use according to the invention is intended for the preparation of pharmaceutical compositions for the treatment of a pathologic disorder associated with heparanase glycosidase catalytic activity, such as a malignant proliferative disorder. For example, a malignant proliferative disorder may be any one of solid and non-solid tumor selected from the group consisting of carcinoma, melanoma, leukemia, and lymphoma.

Still further, the use according to the invention, may be applicable for the preparation of pharmaceutical compositions for the treatment of a pathologic disorder associated with heparanase glycosidase catalytic activity, that may be an inflammatory disorder or an autoimmune disorder.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It must be noted that, as used in this specification and the appended claims, the singular forms “a”, “an” and “the” include plural referents unless the content clearly dictates otherwise.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Experimental Procedures

Cells

Human Eosinophil cells were purified from the peripheral blood of mildly atopic volunteers according to the guidelines established by the Hadassah-Hebrew University Human Experimentation Helsinki Committee [Levi-Schaffer (1999) ibid.].

Human mast cell line HMC-1, used as a sonicate for eosinophil activation (a kind gift from Dr. J. Butterfield, Mayo Clinic, Rochester, Minn.).

Human foreskin fibroblast cell line HS68 (ATCC No CRL 1635, American Type Culture Collection).

Primary bovine aortic endothelial and smooth muscle cells were isolated as described elsewhere [Levi-Schaffer (1999) ibid.; Nagler, A. et al., Arteriosclerosis, Thrombosis & Vascular Biol. 17:194-2002 (1997)].

MBP Isolation and Purification

MBP was isolated as previously described from eosinophils of patients with hypereosinophilic syndrome [Slifman, N. R. et al., J. Immunol. 137:2913-2917 (1986)]. The MBP was pure as analyzed by the presence of a single band after SDS-PAGE and staining with Coomassie Brilliant Blue R250.

Eosinophil Cells Activation

For activation experiments, eosinophils (purity>99%, Kimura's, viability>99%, Trypan blue; Sigma) were resuspended (1×10⁶/ml) in RPMI-1640 supplemented with 200 μg/ml streptomycin, 200 U/ml penicillin, 2 mM gentamicin, 2 mM glutamine, 0.1 mM non-essential amino acids and 5% (v/v) heat-inactivated fetal calf serum (FCS) (Biological Industries, Beit Haemek, Israel) and cultured in 96 well plates in medium alone or with either PAF (1×10⁻⁷ M) (Sigma, St. Louis, Mo.), IL-2 (25 ng/ml) (R&D Systems, Inc. Minneapolis, Minn.), human mast cell line HMC-1 sonicate (1×10⁵/ml), Phorbol Myristate Acetate (PMA, 2.5 ng/ml), or recombinant human skin β-tryptase (50 ng/ml) for 15 min or 18 h as described [Simon H-U. et al., J. Immunol. 165:4069-75 (2000); Temkin, V. et al., J. Immunol. 169:2662-9 (2002); Hoenstein R. et al., Cell. Immunol. 210:116-24 (2001)]. Eosinophils were also pre-incubated (20 min) in medium alone or in GM-CSF (50 ng/ml) (R&D Systems, Inc. Minneapolis, Minn.), followed by C5a stimulation (1×10⁻⁸ M) (R&D Systems, Inc. Minneapolis, Minn.) (25 min) [Simon (2000) ibid.]. EPO release was evaluated by an enzymatic colorimetric assay [White, R. et al., J. Immunol. Methods 44: 257-63 (1991)] and IL-6 by a commercial ELISA assay in the supernatants. For lysate preparation, eosinophils and neutrophils (1×10⁶/ml) were resuspended in RPMI without FCS, adjusted to pH 6.0 by 20 mM buffer citrate phosphate and lysed by 3 cycles of freezing and thawing [Matzner (1985) ibid.].

For co-culture studies, confluent human foreskin fibroblast cell line HS68 and primary bovine aortic endothelial and smooth muscle cells (3-5 passages) seeded in 50 ml flasks (Costar, Cambridge, Mass.) were incubated with sonicated [Levi-Schaffer (1999) ibid.] or viable eosinophils (1×10⁶/flask) in the presence of GM-CSF (10 ng/ml).

Heparanase Activity Assay

Cell-associated heparanase activity was determined as described [Vlodavsky (1999 (a)) ibid.; Vlodavsky (1992) ibid.; Vlodavsky, I. et al., Invasion Metastasis 14:290-302 (1994)]. Briefly, metabolically Na₂[³⁵S]O₄-labeled ECM, firmly bound to the tissue culture plastic and free of cellular debris [Vlodavsky, I. et al., Current Protocols in Cell Biology. Vol 1. New York, N.Y.: John Wiley & Sons; p. 10.14.11-10.14.14 (1999(b))], was incubated (4 h, 37° C., pH 6.0) with cell lysates (eosinophils, neutrophils, fibroblasts), or with eosinophil supernatants, or MBP, EPO, ECP or human recombinant heparanase [Zetser et al., Can. Res. 63:7733-7741 (2003); Gong et al. J. Biol. Chem. 278:35152-35158 (2003)] in heparanase reaction mixture (50 mM NaCl, 1 mM CaCl₂, 1 mM DTT and 20 mM citrate-phosphate buffer, pH 6.0). In order to evaluate the presence of HS degradation products, the incubation medium was applied onto CL-Sepharose 6B columns (0.5×30 cm) [Vlodavsky (1999(a)) ibid.; Vlodavsky (1992) ibid.].

The inventors have previously demonstrated that nearly 80% of the ECM sulfate labeled material is incorporated into HS proteoglycans and that heparanase releases from the ECM degradation fragments of HS that are eluted at 0.5<kₐᵥ<0.8 (fractions 15-35, peak II). Nearly intact HS proteoglycans were next eluted to the V₀ (kₐᵥ<0.2, peak I). Each experiment was performed at least three times, and the variation in elution positions (kₐᵥ values) did not exceed±15%.

Immunocytochemistry and Confocal Microscopy Analysis

Cytospins (5 min, 1000 g) of freshly isolated eosinophils (1×10⁵) were fixed in methanol (3 min, −20° C.) and stained with anti-human heparanase monoclonal antibodies (mAb-130, 10 μg/ml, 2 h, 24° C.) (kindly provided by InSight Ltd., Rehovot, Israel), followed by secondary antibodies, using a ZIMED kit (ZIMED Laboratories Inc, San Francisco, Calif.) [Friedman, Y. et al., Am. J. Pathol. 157: 1167-75 (2000)]. For co-staining and confocal microscopy, either rabbit anti-human MBP IgG (0.5 μg/ml, 12 h, 24° C.) and Cy5 goat anti-rabbit fluorescent secondary antibodies, or anti-heparanase mAb-130 followed by Cy2 goat anti-mouse antibodies (Jackson ImmunoResearch Laboratories, Inc., West Grove, Pa.) were added [Goldshmidt, O. et al., Exp. Cell Res. 281:50-62 (2002)]. Cells were examined and visualized as described [Temkin (2002) ibid.].

RNA Isolation and RT-PCR

RNA was isolated from freshly isolated eosinophils (2×10⁶) with Tri-Reagent (Sigma, St. Louis, Mo.) and quantified [Vlodavsky (1999(a)) ibid.]. Following reverse transcription (RT) of 2 μg total RNA by oligo (dT) 17 mer priming (GIBCO, BRL, Life Technologies, Rockville, Md.), the resulting single stranded cDNA was amplified using Taq DNA Polymerase (Promega, Madison, Wis.) and dNTP mixture [Vlodavsky (1999(a)) ibid.] Human heparanase specific oligonucleotide primers; 5′-GCAAAACTCTATGGTCC TGATGT-3′ (also denoted by SEQ ID NO: 1) and 5′-GCAAAGGTGTCGGATAGCMG-3′ (also denoted by SEQ ID NO: 2), yielding a 299 bp product, were used. The PCR conditions were: denaturation (94° C., 2 min; 94° C., 15 s), annealing (45 s, 58° C.), extension (1 min, 72° C.) (33 cycles) [Vlodavsky (1999(a)) ibid.].

Aliquots (15 μl) of the amplified cDNA were separated by 1.5% agarose gel electophoresis and visualized by ethidium bromide staining. A cDNA template of human heparanase was used as a positive control [Vlodavsky (1999(a)) ibid.; Goldshmidt, O. et al., J. Biol. Chem. 276:29178-87 (2001)]. Only RNA samples that gave completely negative results in PCR without transcriptase were analyzed.

Western Blot and Immunoprecipitation

For immunoblot analysis, lysates were mixed with heparin-Sepharose beads and incubated (1 h, 24° C.) with rotation. The beads were washed twice (PBS), boiled with Laemmli buffer and centrifuged. The supernatants were separated by 10% SDS-PAGE. The proteins were transferred from the gel to Immobilon-P membrane (Millipore, Bradford, Mass.) that was sequentially incubated with block solution, anti-human heparanase monoclonal antibodies and horseradish peroxidase-conjugated rabbit anti-mouse antibodies (DAKO Corporation, Carpinteria, Calif.) [Vlodavsky (1999(a)) ibid.; Friedman (2000) ibid.; Goldshmidt (2001) ibid.]. ECL visualization was performed with SuperSignal West Pico Trial Kit (Pierce) and a subsequent exposure to Fuji film Super RX for 10-60 sec.

For immunoprecipitation, protein A-Sepharose beads were first saturated with anti-human MBP IgGs and incubated (1 h, 24° C.) with cell (eosinophils, fibroblasts) lysates. The Protein A Sepharose-MBP-heparanase complex was then boiled in Laemmli buffer, and subjected to Western blot analysis, as described above.

Allergic Peritonitis in TNF-Knockout Mice

Allergic peritonitis was induced in wild type male C57BL/6 (WT) and C57BL/6 TNF-knockout (TNF-KO) mice [Temkin V. et al., Cytokine (2003) in Press] (a gift from Prof. H. P. Eugster, Department of Pathology, University of Bern, Bern, Switzerland), 8 weeks old and weighing 25-30 g, with ovalbumin (OVA), as described [Temkin (2003) ibid.].

The experimental protocols were approved by the Animal Experimentation Committee of The Hebrew University of Jerusalem. Mice were sacrificed 3 days after challenge and the peritoneal cavity was washed with 5 ml of Tyrode buffer containing 0.1% gelatin (TG buffer) [Temkin (2003) ibid.]. Peritoneal lavage fluid was centrifuged (5 min, 150 g), supernatants were saved for heparanase assessment and cell pellets resuspended in 2 ml of TG buffer for eosinophil quantification [Temkin (2003) ibid.].

Experimental Metastasis

Six week-old male C57BL/6 mice were injected into the lateral tail vein with 0.4 mL of cell suspention containing 0.4×10⁶ B-16 melanoma cells that were incubated for 15 min in the presence of isolated and purified MBP (180 μg/ml) or saline (control) prior to their injection. Sixteen days after cell injection, mice were scarified, their lungs removed, fixed in Bouin's solution, and scored for the number of metastatic nodules on the lung surface under a dissecting microscope.

### Example 1

Evaluation of Heparanase Expression and Localization in Eosinophils

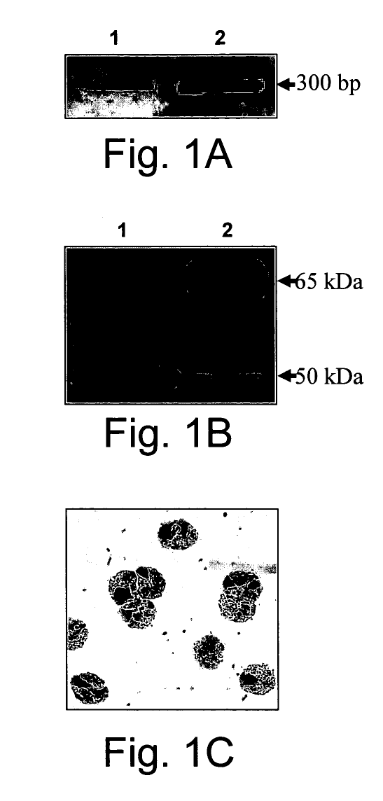

The increased significant role ascribed recently for heparanase in inflammation, angiogenesis and cancer progression, have led the inventors to investigate the possible involvement of heparanase in inflammation and allergy processes mediated by eosinophils. As a first step, the expression of heparanase mRNA in freshly isolated human peripheral blood eosinophils was demonstrated by RT-PCR using heparanase specific primers (FIG. 1A). Both the processed (50 kDa) and, to a lesser extent, the unprocessed (65 kDa) forms of heparanase were detected by Western blot analysis of lysed freshly isolated eosinophils (FIG. 1B, lane 1). On the basis of this experiment, the inventors estimated that 1×10⁶ eosinophils contain 2-4 ng of heparanase similar to other cells of the immune system [Vlodavsky (1992) ibid.; Matzner (1985) ibid.]. By staining the eosinophils with anti-human heparanase monoclonal antibodies it was found that all the examined cells contained preformed heparanase in their cytoplasm, appearing in a granular pattern (FIG. 1C).

Co-localization of Heparanase with MBP in Eosinophil Cells

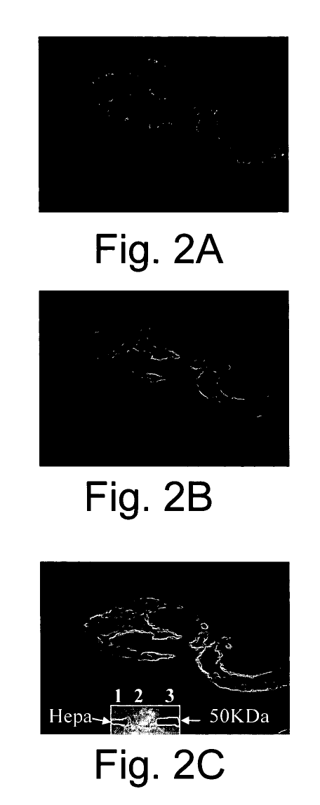

Confocal microscopy analysis of the eosinophils demonstrated that heparanase (FIG. 2B, green Cy2) partly co-localized with MBP (FIG. 2A, red CY2) in overlapping distinct yellow regions (FIG. 2C). Consequently, to evaluate the possible interaction between heparanase and MBP, eosinophil lysates were incubated with anti-MBP antibodies, precipitated with protein A-Sepharose beads and subjected to SDS/PAGE followed by immunoblot analysis with anti-heparanase antibodies. As shown (FIG. 2C inset), the immunoprecipitate from eosinophils (lane 1) contained a 50 kDa protein, corresponding to recombinant human heparanase (lane 3, used as positive control), while human foreskin fibroblasts, used as a negative control, did not (lane 2).

### Example 2

Evaluation of Eosinophil-Associated Heparanase Enymatic Activity

Next, the inventors evaluated whether eosinophils display heparanase enzymatic activity. For this purpose, lysates of freshly isolated eosinophils were incubated with sulfate labeled ECM. These samples failed to release sulfate labeled HS degradation fragments (FIG. 3), indicating a lack of heparanase enzymatic activity. In contrast, lysed neutrophils exhibited a high heparanase activity, releasing 60-70% of the total ECM incorporated radioactivity in the form of HS degradation fragments (fractions 20-35) (FIG. 3). The biochemical nature of these cleavage fragments was characterized in previous studies [Matzner (1985) ibid.]. Subsequently, the inventors tried to induce heparanase activity by activating the eosinophils for 15 min. or 18 h, with either PAF, PMA, recombinant human skin α-tryptase, IL-2 or sonicated HMC-1 cells. As observed with resting cells, none of these treatments induced heparanase activity, measured either in the eosinophil cell lysates or in their supernatants, even though they elicited eosinophil activation, as detected by EPO and/or IL-6 release (not shown) [Simon (2000) ibid.; Temkin (2002) ibid.; Hoenstein (2001) ibid.]. Even activation achieved by incubating the eosinophils with GM-CSF and C5a [Simon (2000) ibid.], that caused a 6.14 fold increase in EPO release over the control (FIG. 4A), did not result in secreted heparanase activity (FIG. 4B). Likewise, co-culture of eosinophils with either endothelial cells, smooth muscle cells or fibroblasts, or the addition of eosinophil sonicate to fibroblasts, failed to yield an enzymatically active heparanase (not shown).

### Example 3

Inhibition of Heparanase Activity by Eosinophils and MBP

The inventors next hypothesized that eosinophils might inhibit heparanase activity. Therefore, the ability of eosinophil lysates to inhibit heparanase-mediated degradation of HS in intact ECM was investigated. For this purpose, active 50 kDa recombinant heparanase (10 ng/ml) was incubated with sulfate labeled ECM in the absence or presence of either lysed eosinophils, or, as a control, lysed human foreskin fibroblasts. As shown in FIG. 5A, release of low-molecular weight labeled HS degradation fragments was specifically abolished by eosinophil lysates, but not by fibroblasts. In a subsequent experiment, the eosinophil lysates were incubated with the labeled ECM in the presence of increasing concentrations of recombinant heparanase. Complete inhibition of activity was obtained even at an heparanase concentration of 1 μg/ml, representing an estimated excess of MBP over heparanase of about 2.5 folds (not shown).

Because of the confocal microscopy showing that heparanase partially co-localizes with MBP (FIG. 2), the inventors assumed that MBP and may be other granular constituents could function as specific inhibitors of heparanase. In fact, when purified MBP was added to recombinant heparanase, a concentration-dependent inhibition of its activity was observed with an almost complete inhibition at 0.8×10⁻⁷ M and a complete inhibition at 2×10⁻⁷ M (FIG. 5B). As shown in FIG. 5C, ECP and EPO also exerted an inhibitory effect, although to a lesser degree than MBP at equimolar concentrations. Since MBP, ECP and EPO share a high cationic charge, the inventors have next tested other highly basic compounds, i.e., compound 48/80 (condensation product of N-methyl-P-methoxyphenethylamine with formaldehyde) [Kuhry, J. G. et al., Agents Actions 16:109-12 (1985)] and myelin basic protein [Chekhonin, V. P. et al., Vopr. Med. Khim. 46:549-63 (2000)]. Even at 20 ng/ml, they did not show any inhibitory effect. A partial inhibition of heparanase activity was exerted by poly-L-arginine at 5 ng/ml and reached an almost complete inhibition at 13 ng/ml (not shown).

### Example 4

Correlation Between Heparanse Activity and Eosinophil Numbers in Murine Allergic Peritonitis

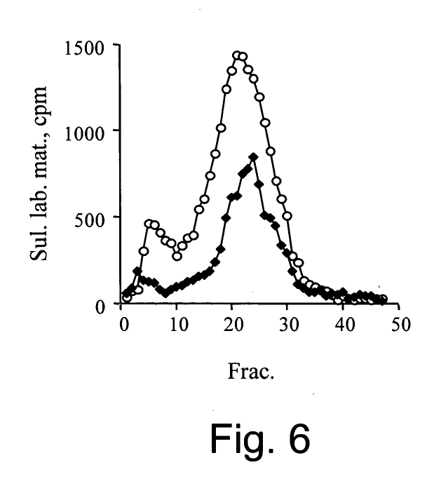

Heparanase activity was assessed in vivo and correlated with eosinophil numbers in TNF-KO and WT mice sensitized and challenged i.p with OVA. As shown in FIG. 6, in TNF-KO mice in which eosinophil numbers were significantly lower than in the WT mice (1.9±0.3×10⁵ cells/ml vs. 5.9±0.7×10⁵ cells/ml, respectively), heparanase activity determined in the peritoneal fluid was 2-3 fold higher.

### Example 5

Inhibition of Melanoma Lung Metastasis by MBP

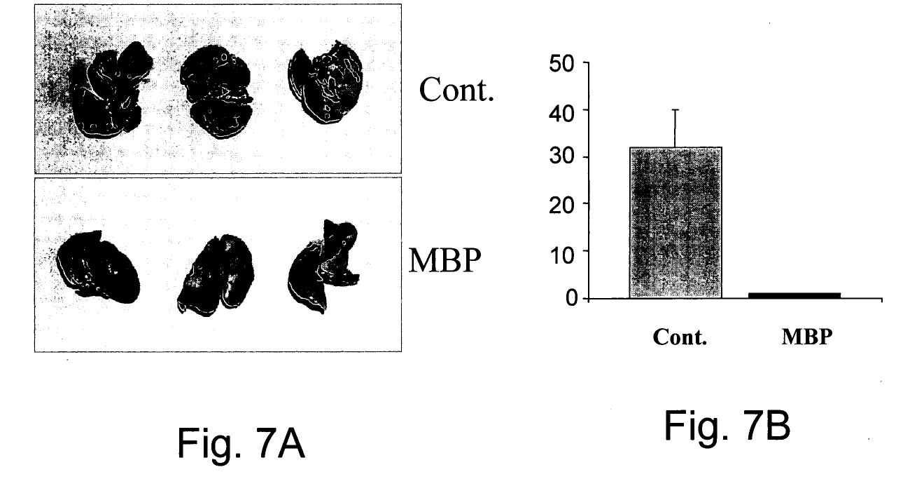

To further evaluate the potential in vivo therapeutic effect of inhibition of heparanase by MBP, a mouse melanoma lung metastasis model was next used by the inventors. It should be noted that this model system was previously used by the inventors to elucidate the direct involvement heparanase in tumor progression [Vlodavsky, et al. Invasion Metastasis 14:290-302 (1994)]. Therefore, B-16 melanoma cells, which are characterized by high levels of endogenous heparanase were incubated for 15 min in the presence of isolated and purified MBP (180 μg/ml) or saline (control), and then injected to the tail vein of C57BL/6 mice (4×10⁵ cells/mouse). Sixteen days later, the mice were scarified, their lungs were excised and evaluated for the number of surface metastatic colonies. As demonstrated by FIG. 7A (lower panel), incubation of the cells with MBP prior to injection, significantly inhibited lung colonization of B-16 melanoma cells. The effect of MBP is clearly shown by the schematic presentation of the number of metastatic lesions in the lungs of mice inoculated with saline (31±8 colonies/lung) vs. MBP (2±0.3 colonies/lung) treated cells, shown in FIG. 7B.

These data clearly demonstrate that specific inhibition of endogenous heparanase by MBP, effectively inhibits the invasive and metastatic potential of B16 melanoma cells.

1-37. (canceled)

38. A composition for the inhibition of heparanase glycosidase catalytic activity, consisting essentially of, as a first constituent, a pharmaceutically acceptable material selected from the group consisting of a carrier, a diluent, an excipient and an additive, and, as a second constituent one of eosinophil cell lysate, eosinophil secondary granules basic protein, poly-L-arginine and mixtures thereof, wherein the second constituent is present in a concentration of about from 1 to about 180 μg/ml.

39. A composition according to claim 38, wherein the eosinophil secondary granules basic protein is selected from the group consisting of the 117 amino acid residue of MBP (Major Basic Protein), ECP (Eosinophil Cationic Protein), EPO (Eosinophil Peroxidase) and EDN (Eosinophil Derived Neurotoxin).

40. A composition according to claim 39, wherein the eosinophil secondary granules basic protein is the 117 amino acid residue of MBP (Major Basic Protein).

41. A composition according to claim 39, wherein said eosinophil secondary granules basic protein is provided as one of a purified recombinant protein, a fusion protein, a nucleic acid construct encoding for said protein, a host cell expressing said construct, a cell, a cell line, tissue endogeneously expressing said protein and a lysate thereof.

42. A method for the inhibition of heparanase glycosidase catalytic activity in a subject consisting essentially of the step of administering to the subject one of eosinophil cell lysate, an eosinophil secondary granules basic protein and mixtures thereof in a concentration of from about 1 to about 180 μg/ml.

43. The method according to claim 42, wherein the eosinophil secondary granules basic protein is selected from the group consisting of the 117 amino acid residue of MBP (Major Basic Protein), ECP (Eosinophil Cationic Protein), EPO (Eosinophil Peroxidase) and EDN (Eosinophil Derived Neurotoxin).

44. The method according to claim 43, wherein the eosinophil secondary granules basic protein is the 117 amino acid residue of MBP (Major Basic Protein).

45. The method according to claim 43, wherein the eosinophil secondary granules basic protein is provided as any one of a purified recombinant protein, a fusion protein, a nucleic acid construct encoding for said protein, a host cell expressing said construct, a cell, a cell line and a tissue endogeneously expressing said protein or a lysate thereof.

46. Method for preparation of a composition for the inhibition of heparanase glycosidase catalytic activity consisting essentially of the step of formulating a first constituent composed of a pharmaceutically acceptable material selected from the group counting of a carrier, a diluent, an excipient and an additive with a second constituent composed of one of eosinophil cell lysate, an eosinophil secondary granules basic protein, and mixtures thereof in a concentration of from about 1 to about 180 μg/ml.

47. The method according to claim 46, wherein the eosinophil secondary granules basic protein is 117 amino acid residue of MBP (Major Basic Protein).

48. Claim The method according to claim 46, wherein the eosinophil secondary granules basic protein is one of a purified recombinant protein, a fusion protein, a nucleic acid construct encoding for said protein, a host cell expressing said construct, a cell, a cell line, a tissue endogeneously expressing said protein and a lysate thereof.

49. A method for the inhibition of heparanase glycosidase catalytic activity consisting essentially of the step of contacting cells having heparanase glycosidase catalytic activity with one of eosinophil cell lysate, an eosinophil secondary granules basic protein and mixtures thereof in a concentration of from about 1 to about 180 μg/ml.
